(19) United States
(12) Patent Application Publication
(10) Pub. No.: US 2023/0256202 A1
(43) Pub. Date: Aug. 17, 2023

(54) STEERABLE MEDICAL DEVICE, HANDLE FOR A MEDICAL DEVICE, AND METHOD FOR OPERATING A MEDICAL DEVICE

(71) Applicant: Boston Scientific Medical Device Limited, Galway (IE)

(72) Inventor: Doran Avivi, Toronto (CA)

(21) Appl. No.: 18/304,020

(22) Filed: Apr. 20, 2023

Related U.S. Application Data
(63) Continuation of application No. PCT/IB2021/059375, filed on Oct. 12, 2021.
(60) Provisional application No. 63/093,910, filed on Oct. 20, 2020.

(51) Int. Cl. A61M 25/01

(52) U.S. Cl. CPC A61M 25/0136; A61M 25/0147

(57) ABSTRACT

A steerable medical device includes an elongate tool, and a handle, and a steering assembly. The handle is mounted to the tool and has a handle body and a knob that is rotatable with respect to the handle body. The steering assembly includes at least a first spool housed within the handle body and coupled to the knob, and at least first control wire windable about the first spool and coupled to the tool. Rotation of the knob with respect to the handle body drives rotation of the first spool, rotation of the first spool cause winding of the first control wire about the first spool to tension the control wire, and tensioning of the control wire causes deflection of the tool.

### CROSS-REFERENCE TO RELATED APPLICATIONS

This application is a continuation of and claims the benefit of International Application Number PCT/162021/059375, entitled “STEERABLE MEDICAL DEVICE, HANDLE FOR A MEDICAL DEVICE, AND METHOD FOR OPERATING A MEDICAL DEVICE,” and filed Oct. 12, 2021, which claims the benefit of U.S. Provisional Application No. 63/093,910, entitled “STEERABLE MEDICAL DEVICE, HANDLE FOR A MEDICAL DEVICE, AND METHOD FOR OPERATING A MEDICAL DEVICE,” and filed Oct. 20, 2020, which are hereby incorporated by reference in their entireties.

### FIELD

This document relates to medical devices. More specifically, this document relates to steerable medical devices such as steerable sheaths, catheters, and introducers.

### SUMMARY

The following summary is intended to introduce the reader to various aspects of the detailed description, but not to define or delimit any invention.

Steerable medical devices are disclosed. According to some aspects, a steerable medical device includes an elongate tool, a handle, and a steering assembly. The handle is mounted to the tool and has a handle body and a knob that is rotatable with respect to the handle body. The steering assembly includes at least a first spool housed within the handle body and coupled to the knob, and at least first control wire windable about the first spool and coupled to the tool. Rotation of the knob with respect to the handle body drives rotation of the first spool, rotation of the first spool causes winding of the first control wire about the first spool to tension the first control wire, and tensioning of the first control wire causes deflection of the tool.

In some examples, the steering assembly includes a gear train coupled between the knob and the first spool, to transfer rotation from the knob to the first spool. The knob can include an internal gear, the first spool can include a bevel gear, and the gear train can include a planetary gear engaged with the internal gear, a sun gear coupled to the planetary gear, and an additional bevel gear coupled between the sun gear and the bevel gear.

In some examples, rotation of the knob with respect to the handle body in a first knob rotational direction drives rotation of the first spool in a first spool rotational direction, rotation of the first spool in the first spool rotational direction causes winding of the first control wire about the first spool to tension the first control wire, and tensioning of the first control wire causes deflection of the tool in a first deflectional direction. Furthermore, in some examples, rotation of the knob with respect to the handle body in a second knob rotational direction drives rotation of the first spool in a second spool rotational direction, and rotation of the first spool in the second spool rotational direction causes unwinding of the first control wire from the first spool to release the tension on the first control wire.

In some examples, the steering assembly further includes a second spool housed within the handle body and coupled to the knob, and a second control wire windable about the second spool and coupled to the tool. Rotation of the knob with respect to the handle body in the second rotational direction can drive rotation of the second spool in the second spool rotational direction, rotation of the second spool in the second spool rotational direction can cause winding of the second control wire about the second spool to tension the second control wire, and tensioning of the second control wire can cause deflection of the tool in a second deflectional direction. Furthermore, rotation of the knob with respect to the handle body in the first knob rotational direction can drive rotation of the second spool in the first spool rotational direction, and rotation of the second spool in the first spool rotational direction can cause unwinding of the second control wire from the second spool to release the tension in the second control wire.

In some examples, the tool is a sheath, a catheter, or an introducer.

Handles for medical devices are also disclosed. According to some aspects, a handle for a medical device includes a handle body, a knob that is rotatable with respect to the handle body, and a steering assembly. The steering assembly includes at least a first spool housed within the handle body and coupled to the knob, and at least first control wire windable about the first spool. Rotation of the knob with respect to the handle body drives rotation of the first spool, and rotation of the first spool causes winding of the first control wire about the first spool to tension the first control wire.

In some examples, the steering assembly includes a gear train coupled between the knob and the first spool, to transfer rotation from the knob to the first spool. The knob can include an internal gear, the first spool can include a bevel gear, and the gear train can include a planetary gear engaged with the internal gear, a sun gear coupled to the planetary gear, and an additional bevel gear coupled between the sun gear and the bevel gear.

In some examples, rotation of the knob with respect to the handle body in a first knob rotational direction drives rotation of the first spool in a first spool rotational direction, and rotation of the first spool in the first spool rotational direction causes winding of the first control wire about the first spool to tension the first control wire. Furthermore, rotation of the knob with respect to the handle body in a second knob rotational direction can drive rotation of the first spool in a second spool rotational direction, and rotation of the first spool in the second spool rotational direction can cause unwinding of the first control wire from the first spool.

In some examples, the steering assembly further includes a second spool housed within the handle body and coupled to the knob, and a second control wire that is windable about the second spool. Rotation of the knob with respect to the handle body in the second knob rotational direction can drive rotation of the second spool in the second spool rotational direction, and rotation of the second spool in the second spool rotational direction can cause winding of the second control wire about the second spool to tension the second control wire. Furthermore, rotation of the knob with respect to the handle body in the first knob rotational direction can drive rotation of the second spool in the first spool rotational direction, and rotation of the second spool in the first spool rotational direction can cause unwinding of the second control wire from the second spool to release the tension in the second control wire.

Methods for operating a medical device are also disclosed. According to some aspects, a method for operating a medical device, includes a. rotating a knob of a handle; b. transmitting rotation of the knob to a first spool housed within the handle to cause rotation of the first spool; c. winding a first control wire about the first spool by rotation of the first spool, to cause tensioning of the first control wire; and d. causing deflection of a tool by tensioning of the first control wire.

In some examples, in step b., rotation of the knob is transmitted to the first spool via a gear train housed within the handle.

In some examples, step a. includes rotating the knob in a first knob rotational direction, step b. includes causing rotation of the first spool in a first spool rotational direction, and step d. includes causes deflection of the tool in a first deflectional direction. The method can further include: e. rotating the knob in a second knob rotational direction, f. transmitting rotation of the knob in the second knob rotational direction to the first spool to cause rotation of the first spool in a second spool rotational direction, and g. unwinding the first control wire from the first spool to release the tension on the first control wire.

In some examples, the method further includes: h. transmitting rotation of the knob in the second rotational direction to a second spool housed within the handle to cause rotation of the second spool in the second spool rotational direction, i. winding a second control wire about the second spool by rotation of the second spool in the second spool rotational direction, to cause tensioning of the second control wire, and j. causing deflection of the tool in a second deflectional direction by tensioning of the second control wire.

In some examples, step b. further includes transmitting rotation of the knob in the first rotational direction to the second spool to cause rotation of the second spool in the first spool rotational direction, and step c. further includes unwinding a second control wire from the second spool to release the tension on the second control wire.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example of medical device, showing a tool and a handle of the medical device;

FIG. 2 is a partial perspective view of the medical device of FIG. 1, with a handle body of the handle removed, in order to show an interior of the handle body;

FIG. 3 is a cross-section taken through the knob of the handle of FIGS. 1 and 2; and

FIG. 4 is a side view of a portion of FIG. 2.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are steerable medical devices that include a handle and a tool such as a sheath, a catheter, or an introducer. The handle can enable the user to steer the tool in a desired direction. More specifically, the handle can include a knob that is rotatably coupled to a body of the handle (also referred to herein as a housing of the handle). In operation, rotation of the knob can allow the user to steer the tool by causing deflection of the tool. Rotation of the knob can be converted into deflection of the tool via a steering assembly, which can include one or more spools housed within the handle body, and one or more control wires, which are windable about the spool(s) and coupled to the tool. Rotation of the knob with respect to the handle body can drive rotation of the spool(s), and rotation of the spool(s) can cause winding of the control wire(s) about the spool(s). Winding of the control wire(s) can cause tensioning of the control wire(s), which can in turn cause deflection of the tool. For simplicity, details of control wire(s) and the connection between the control wire(s) and the tool are not disclosed herein. However, related control wires are disclosed in, for example, U.S. Pat. No. 10,661,057 (Davies et al.), which is incorporated herein by reference in its entirety. Furthermore, steerable medical devices including control wires are sold Baylis Medical Company, Inc. (Montreal, Canada) under the brand name SureFlex® Steerable Guiding Sheath.

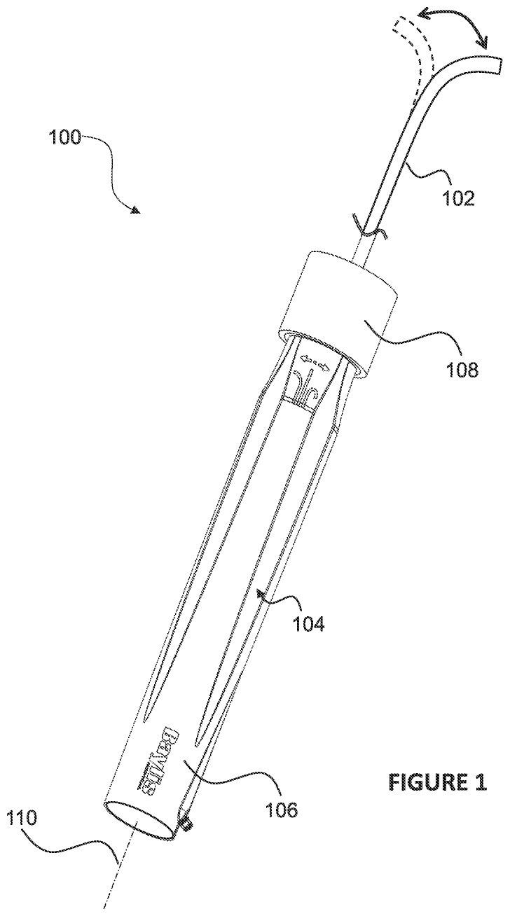

Referring now to FIG. 1, an example steerable medical device 100 is shown. The steerable medical device 100 generally includes an elongate tool 102, and a handle 104 mounted to the tool 102. The tool 102 can be, for example (but not limited to), a sheath, a catheter, or an introducer. The handle 104 can allow the user to grip, manipulate, and steer the tool 102, and includes a handle body 106 and a knob 108 that is rotatable with respect to the handle body 106 to steer the tool 102. Rotation of the knob 108 in a first direction (also referred to herein as a “first knob rotational direction”, e.g. clockwise) can cause the tool 102 to deflect in a first deflectional direction (i.e. to the configuration shown in dotted line in FIG. 1), and rotation of the knob 108 in a second direction (also referred to herein as a “second knob rotational direction”, e.g. counter-clockwise) can cause the tool 102 to deflect in a second deflectional direction (i.e. back to the configuration shown in solid line in FIG. 1).

In the example shown, the handle 104 extends along a handle axis 110, and the knob 108 is rotatable about the handle axis 110 with respect to the handle body 106. In alternative examples, the knob 108 can be rotatable about an axis of rotation that is at another angle with respect to the handle axis, for example perpendicular to the handle axis.

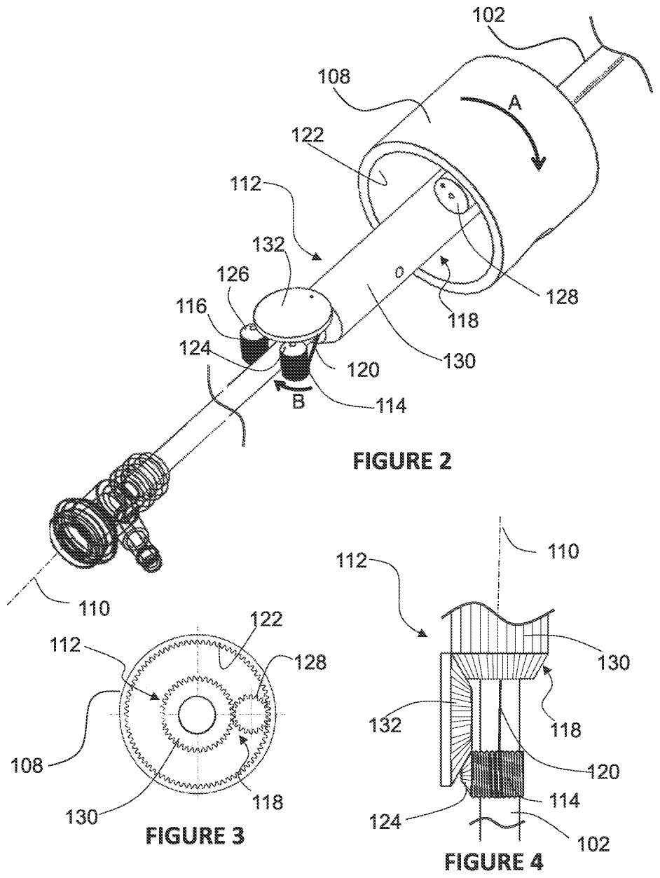

Referring to FIG. 2 (in which the handle body 106 is not shown in order to show the interior thereof) to 4, a steering assembly 112 is provided for converting rotation of the knob 108 into deflection of the tool 102. As will be described in greater detail below, the steering assembly 112 includes a first spool 114 and a second spool 116 (shown only in FIG. 2), which are housed within the handle body 106 and coupled to the knob 108 via a gear train 118 (described in greater detail below with respect to FIG. 3), and a first control wire 120 and a second control wire (not visible), which are windable about the first spool 114 and second spool 116, respectively. As will be described in greater detail below, rotation of the knob 108 drives rotation of the first spool 114 and the second spool 116. Rotation of the first spool 114 causes winding of the first control wire 120 about the first spool to tension the first control wire 120, and rotation of the second spool 116 causes winding of the second control wire about the second spool 116 to tension the second control wire. Tensioning of the first 120 and second control wires causes deflection of the tool 102, in the first and second deflectional directions, respectively.

Referring to FIGS. 3 and 4, as mentioned above, the knob 108 is coupled to the first spool 114 and second spool 116 via a gear train 118. The gear train 118 is coupled between the knob 108 and the first 114 and second 116 spools to transfer rotation from the knob 108 to the first 114 and second 116 spools. In the example shown, the knob 108 includes an internal gear 122 (shown in FIG. 3), each spool 114, 116 includes a respective bevel gear 124, 126 (the bevel gear 126 of the second spool 116 is shown in FIG. 2), and the gear train 118 includes a planetary gear 128 engaged with the internal gear 122, a sun gear 130 coupled to the planetary gear 128, and an additional bevel gear 132 coupled between the sun gear 130 and the bevel gears 124 and 126.

Referring back to FIG. 2 (in which the teeth of the gears are not shown) rotation of the knob 108 with respect to the handle body 106 in a first direction (also referred to herein as “a first knob rotational direction”, and as shown by arrow A in FIG. 2) drives rotation of the first spool 114 and the second spool 116 in a first direction (also referred to herein as “a first spool rotational direction”, and shown by arrow B in FIG. 2), via the gear train 118. Referring to FIG. 4, rotation of the first spool 114 in the first rotational direction causes winding of the first control wire 120 about the first spool 114, while rotation of the second spool 116 in the first rotational direction causes unwinding of the second control wire from the second spool 116 (not shown). Winding of the first control wire 120 about the first spool 114 causes tensioning the first control wire 120, while unwinding of the second control wire releases tension in the second control wire. Tensioning of the first control wire 120, with the tension released in the second control wire, causes deflection of the tool 102 in the first deflectional direction (i.e. to the configuration shown in dotted line in FIG. 1).

Referring back to FIG. 2, rotation of the knob 108 with respect to the handle body 106 in the opposite direction (also referred to herein as “a second knob rotational direction”) drives rotation of the first spool 114 and the second spool 116 in the opposite direction (also referred to herein as “a second spool rotational direction), via the gear train 118. Referring to FIG. 4, rotation of the first spool 114 in the second spool rotational direction causes unwinding of the first control wire 120 from the first spool 114, while rotation of the second spool 116 in the second spool rotational direction causes winding of the second control wire about the second spool 116 (not shown). Unwinding of the first control wire 120 from first spool 114 releases the tension on the first control wire 120, while winding of the second control wire about the second spool 116 causes tensioning of the second control wire. Tensioning of the second control wire, with the tension released in the first control wire 120, causes deflection of the tool 102 in the second deflectional direction (i.e. to the configuration shown in solid line in FIG. 1).

In the example shown, the steering assembly 112 includes two spools and two control wires. In alternative examples the steering assembly can include another number of spools and control wires, such as one spool and one control wire.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A steerable medical device comprising:
an elongate tool;
a handle mounted to the tool and having a handle body and a knob that is rotatable with respect to the handle body; and
a steering assembly comprising at least a first spool housed within the handle body and coupled to the knob, and at least first control wire windable about the first spool and coupled to the tool, wherein rotation of the knob with respect to the handle body drives rotation of the first spool, rotation of the first spool causes winding of the first control wire about the first spool to tension the first control wire, and tensioning of the first control wire causes deflection of the tool.

2. The steerable medical device of claim 1, wherein the steering assembly comprises a gear train coupled between the knob and the first spool, to transfer rotation from the knob to the first spool.

3. The steerable medical device of claim 1, wherein the knob comprises an internal gear, the first spool comprises a bevel gear, and the gear train comprises a planetary gear engaged with the internal gear, a sun gear coupled to the planetary gear, and an additional bevel gear coupled between the sun gear and the bevel gear.

4. The steerable medical device of claim 1, wherein rotation of the knob with respect to the handle body in a first knob rotational direction drives rotation of the first spool in a first spool rotational direction, rotation of the first spool in the first spool rotational direction cause winding of the first control wire about the first spool to tension the first control wire, and tensioning of the first control wire causes deflection of the tool in a first deflectional direction.

5. The steerable medical device of claim 4, wherein rotation of the knob with respect to the handle body in a second knob rotational direction drives rotation of the first spool in a second spool rotational direction, rotation of the first spool in the second spool rotational direction causes unwinding of the first control wire from the first spool to release the tension on the first control wire.

6. The steerable medical device of claim 5, wherein the steering assembly further comprises a second spool housed within the handle body and coupled to the knob, and a second control wire windable about the second spool and coupled to the tool, wherein rotation of the knob with respect to the handle body in the second rotational direction drives rotation of the second spool in the second spool rotational direction, rotation of the second spool in the second spool rotational direction cause winding of the second control wire about the second spool to tension the second control wire, and tensioning of the second control wire causes deflection of the tool in a second deflectional direction.

7. The steerable medical device of claim 6, wherein rotation of the knob with respect to the handle body in the first knob rotational direction drives rotation of the second spool in the first spool rotational direction, and rotation of the second spool in the first spool rotational direction cause unwinding of the second control wire from the second spool to release the tension in the second control wire.

8. The steerable medical device of claim 1, wherein the tool is a sheath, a catheter, or an introducer.

9. A handle for a medical device, the handle comprising:
a handle body and a knob that is rotatable with respect to the handle body; and
a steering assembly comprising at least a first spool housed within the handle body and coupled to the knob, and at least first control wire windable about the first spool, wherein rotation of the knob with respect to the handle body drives rotation of the first spool, rotation of the first spool cause winding of the first control wire about the first spool to tension the first control wire.

10. The handle of claim 9, wherein the steering assembly comprises a gear train coupled between the knob and the first spool, to transfer rotation from the knob to the first spool.

11. The handle of claim 10, wherein the knob comprises an internal gear, the spool comprises a bevel gear, and the gear train comprises a planetary gear engaged with the internal gear, a sun gear coupled to the planetary gear, and an additional bevel gear coupled between the sun gear and the bevel gear.

12. The handle of claim 9, wherein rotation of the knob with respect to the handle body in a first knob rotational direction drives rotation of the first spool in a first spool rotational direction, and rotation of the first spool in the first spool rotational direction cause winding of the first control wire about the first spool to tension the first control wire.

13. The handle of claim 12, wherein rotation of the knob with respect to the handle body in a second knob rotational direction drives rotation of the first spool in a second spool rotational direction, and rotation of the first spool in the second spool rotational direction causes unwinding of the first control wire from the first spool.

14. The handle of claim 13, wherein:
the steering assembly further comprises a second spool housed within the handle body and coupled to the knob, and a second control wire windable about the second spool; and
rotation of the knob with respect to the handle body in the second rotational direction drives rotation of the second spool in the second spool rotational direction, and rotation of the second spool in the second spool rotational direction causes winding of the second control wire about the second spool to tension the second control wire.

15. The handle of claim 14, wherein rotation of the knob with respect to the handle body in the first knob rotational direction drives rotation of the second spool in the first spool rotational direction, and rotation of the second spool in the first spool rotational direction cause unwinding of the second control wire from the second spool to release the tension in the second control wire.

16. A method for operating a medical device, comprising:
a. rotating a knob of a handle;
b. transmitting rotation of the knob to a first spool housed within the handle to cause rotation of the first spool;
c. winding a first control wire about the first spool by rotation of the first spool, to cause tensioning of the first control wire;
d. causing deflection of a tool by tensioning of the first control wire.

17. The method of claim 16, wherein in step b., rotation of the knob is transmitted to the first spool via a gear train housed within the handle.

18. The method of claim 16, wherein:
step a. comprises rotating the knob in a first knob rotational direction;
step b. comprises causing rotation of the first spool in a first spool rotational direction; and
step d. comprises causes deflection of the tool in a first deflectional direction; and
the method further comprises:
e. rotating the knob in a second knob rotational direction,
f. transmitting rotation of the knob in the second rotational direction to the first spool to cause rotation of the first spool in a second spool rotational direction,
g. and unwinding the first control wire from the first spool to release the tension on the first control wire.

19. The method of claim 18, wherein the method further comprises:
h. transmitting rotation of the knob in the second rotational direction to a second spool housed within the handle to cause rotation of the second spool in the second spool rotational direction; and
i. winding a second control wire about the second spool by rotation of the second spool in the second spool rotational direction, to cause tensioning of the second control wire, and causing deflection of the tool in a second deflectional direction by tensioning of the second control wire.

20. The method of claim 19, wherein:
step b. further comprises transmitting rotation of the knob in the first rotational direction to the second spool to cause rotation of the second spool in the first spool rotational direction; and
step c. further comprises unwinding the second control wire from the second spool to release the tension on the second control wire.
